(19) United States
(12) Patent Application Publication
(10) Pub. No.: US 2008/0103116 A1
(43) Pub. Date: May 1, 2008

(54) Method of treatment and compositions of D-chiro inositol and phosphates thereof

(76) Inventor: Barbara L. Jennings-Spring, Jupiter, FL (US)

Correspondence Address: Irving M. Fishman;c/o Cohen, Tauber Spievack & Wagner, Suite 2400, 420 Lexington Avenue, New York, NY, 10170, US

(21) Appl. No.: 11/591,398

(22) Filed: Nov. 1, 2006

(51) Int. Cl. A61K 31/66; A61K 31/56; A61K 31/525; A61K 31/045

(52) U.S. Cl. 514/102; 514/251; 514/729; 514/171

(57) ABSTRACT

The present invention relates to the use of D-chiroinositol or a phosphate thereof in combination with folate for the reduction or prevention of congenital deformations such as anorectal malformations, neural tube defects, cleft-lip, cleft palate, and other birth defects. The invention further relates to the use of D-chiroinositol or a phosphate thereof in quieting or preventing the sensitivity of breast tissue to estrogenic, progestogenic, and or anti-androgenic insult, whether from environmental, dietary, or medicinal sources. Co-therapies as well as combination products of D-chiro-inositol (or a phosphate thereof) with at least one of (a) a folate source and (b) one or more of an estrogenic substance, a progestogenic substance, and/or an antiandrogenic substance are also claimed.

### CROSS-REFERENCE TO RELATED APPLICATIONS

Not Applicable

### STATEMENT REGARDING FEDERALLY SPONSORED RESEARCH OR DEVELOPMENT

Not Applicable

### FIELD OF THE INVENTION

The present invention relates to the field of fetal malformations and birth defects. It further relates to chiroinositol and phosphates thereof, more specifically D-chiroinositol and phosphates thereof. In addition, the present invention relates to folates. The present invention further relates to downregulation of estrogenic sensitive breast tissue to exposure to estrogenic substances or estrogenic surplus. The invention also relates to co-therapy methods and sequential treatment regimens relating to the above and to compositions for the prevention and/or minimization of fetal malformations and for the prevention, minimization, and/or treatment of the sequela of estrogen exposure or estrogen surplus exposure of estrogen-receptor positive breast tissue.

### BACKGROUND OF THE INVENTION

Fetal malformations are a continuing medical problem in serious need of prevention and treatment. These malformations can result in innocuous defects that pose no health or psychological issues, to those that pose primarily social or psychological issues (such as webbed digits, etc.), to those that pose medical issues of varying degrees of severity. Some of the more medically severe malformations include neural tube defects (such as, among others, anencephaly where the brain is underdeveloped or there is an incomplete skull, encephalocele, where there is a hole in the skull through which tissue protrudes, and spina bifida, where a portion of the spine is exposed) to cranio-facial defects (such as, among others, cleft lip and cleft palate) to imperforate anus (where the anal opening doesn't form properly leaving no exit for intestinal contents, or intestinal/rectal emptying into inappropriate structures such as the bladder, ureter, uterus or vagina).

The number of births presenting with spina bifida has been reduced in recent years in patients at risk of having such defects by having adequate folate levels in the mother just before and during the first trimester of pregnancy. More specifically, if a woman takes folic acid before conception and during early pregnancy, the risk of the fetus developing a neural tube defect is reduced by about 70%. Unfortunately, folate supplementation still does not prevent all such cases, and the remaining 30% risk is still substantial. In a Research Review from Neurosciences and Mental Health 2005 from Great Ormond Street Hospital, the use of inositol in combination with folate therapy is mentioned as being explored. The Review indicates that initial findings in women who took inositol during pregnancy is encouraging and that formal clinical trials involving women having had a baby with a neural tube defect and planning another baby are to be given folic acid or a combination of folic acid and inositol to determine if the combination is beneficial. However, no particular type of inositol is mentioned nor is any dosage amount or regimen.

Inositol prevents expression of a genetic model of neural tube defects in mice; Nutrition Reviews, May 1997 reports that myo-inositol reduced the incidence of neural tube defects in mouse models that are folate resistant. In Cogram et al, D-chiro-inositol is more effective than myo-inositol in preventing folate-resistant mouse neural tube defects; Human Reproduction, Vol. 17, No. 9, 2451-2458, the investigators found that the D-chiro form of inositol was better at preventing neural tube defects in the ‘curly tail’ mouse model than myo-inositol. The curly tail model is particularly resistant to folate therapy. Cogram states that while both D-chiro-inositol and myo-inositol reduced frequency of spina bifida in this model, the D-chiro-inositol group had a 73-86% reduction vs a 53-56% reduction for the myo-inositol group, and thus raised the possibility that D-chiro-inositol as an adjunct to folic acid for the prevention of neural tube defects.

Meyers, et al; Folic Acid Supplementation and Risk for Imperforate Anus in China; American Journal of Epidemiology, Vol. 154, No. 11: 1051-1056, 2001 reports on a public health campaign in China in 1993 to 1995, where women were requested to take 400 mg folic acid, with or without other vitamins daily from their pre-marital examination through the end of their first trimester of pregnancy. The rate of imperforate anus was calculated to be 3.1 per 10,000 births for those not taking folic acid compare to 1.6 per 10,000 births for those taking folic acid. The authors conclude that folic acid may reduce imperforate anus risk.

Inositols are a group of compounds that have the following structure:

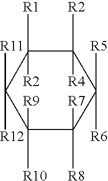

where each of the R groups is either H or OH, but each carbon of the ring has one H and one OH. The most common form is myo-inositol, which is available to some degree from dietary sources. Myo-inositol requires that all of R1, R3, R5, R8, R9, and R12 are OH and R2, R4, R6, R7, R10, and R11 are all hydrogen. Epi-inositol and scyllo-inositol are the other two most abundant forms (each being substantially less than the myo-inositol in terms of abundance). D-chiro-inositol is not available from dietary sources and is the isomer where R1, R3, R6, R8, R9, and R12 are OH and R2, R4, R5, R7, R10, and R11 are hydrogen. In other words, D-chiro-inositol differs from myo-inositol in the inversion of R5/R6.

There are a total of eight isomers of inositol, and for those that have found potential medicinal or nutritional use, many of the uses are truly limited to particular isomers and/or phosphates (where one or more of the hydroxyl groups are phosphorylated) thereof, while for other uses more than one inositol isomer has been found useful or is projected to be useful. For example, recently scyllo inositol has been found to prevent the accumulation of amyloid P deposits and improved cognitive ability in Alzheimer's patients. (McLaurin, et al, Inositol Stereoisomers Stabilize an Oligomeric Aggregate of Alzheimer Amyloid beta Peptide and Inhibit A beta-induced Toxicity, J. Biol. Chem., Vol. 275, Issue 24, 18495-18502, Jun. 16, 2000; and Research News from Howard Hughes medical Institute Jun. 11, 2006 A Sweet Solution to Alzheimer's Disease?) Myo-inositol was found not to be effective in this condition. Scyllo-inositol worked when given before symptoms appeared as well as after symptoms appeared in this indication, while epi-inositol only worked at all when given before disease onset. Interestingly, scyllo-inositol has been reported to be an “inositol” uptake inhibitor causing similar fetal development defects in non-hyperglycemic pregnancies as seen in hyperglycemic pregnancies (Cederberg; Oxidative Stress, antioxidative defense, and Outcome in Experimental Diabetic pregnancy, Comprehensive Summaries of Uppsala Dissertations from the Faculty of medicine 1008, AUU Uppsala 2001, pp. 1-66). Myo-inositol has been found useful in treating panic attacks (Levine, et al, Double-blind, placebo-controlled, crossover trial for inositol treatment for panic disorder, Am J Psychiatry 1995; 152; 1084-1086). Cleft palate children were found to have low red blood cell zinc levels and low myo-inositol levels (Krapels, et al: Myo-inositol, glucose and zinc status as risk factors for non-syndromic cleft lip with or without cleft palate in offspring: a case-control study, BJOG. 2004 July; 111(7):661-8) although there is no indication if the low myo-inositol level is a cause, result, or merely coincidental with the presentation of the defect. Inositol hexaphosphate has been found to have anti-cancer activity (Vucenik et al Cancer Inhibition by Inositol Hexaphosphate (IP₆) and Inositol: From Laboratory to Clinic, J. Nutr. 133:3778S-3784S, November 2003) and further U.S. Pat. No. 5,082,833 (which, along with all other patents mentioned in this disclosure is incorporated herein by reference in its entirety) discloses combination thereof with inositol has been found to boost that effect. Myo-inositol and epi-inositol have been found to reverse lithium-pilocarpine seizures.

One of the more prominent uses for myo-inositol has been for blood sugar regulation. Recently, D-chiro-inositol has been proposed for insulin resistance patients (Larner, D-Chiro-Inositol—Its functional role in Insulin Action and its Deficit in Insulin Resistance, International Journal of Experimental Diabetes Research 3 (2002), 47-60) on the theory that such patients have a defect in epimerization of the myo-inositol to the D-chiro-inositol and that the D-chiro-inositol is the active moiety in this regard. As stated above, scyllo-inositol actually resulted in an increase in fetal defects in non-hyperglycemic pregnancy similar to that seen in hyperglycemic pregnancy. Thus, it is clear that an activity demonstrated by one isomer of inositol is not automatically shared or expected to be shared by another isomer of inositol.

An excellent review of inositol and some of its phosphates is given in Fisher, et al; Inositol and higher inositol phosphates in neural tissues: homeostasis, metabolism and functional significance; Journal of Neurochemistry, Vol 82, 736 August 2002. Other relevant literature includes: Frederick, et al; An essential role for an inositol polyphosphate multikinase, Ipk2, in mouse embryogenesis and second messenger production, PNAS Jun. 14, 2005, Vol 102, No. 24, 8454-8459; Riobo, et al Phosphoinositide 3-kinase and Akt are essential for sonic Hedgehog signaling, PNAS Mar. 21, 2006, Vol. 103, No. 12, 4505-4510. In addition, Mo et al, Anorectal malformations Caused by Defects in Sonic Hedgehog signaling, American Journal of Pathology 2001, 159, 765-774 report on a mutant mouse with various defects in the Sonic Hedgehog signaling pathway that presents with a number of distal hindgut defects that appear to the authors to mimic human anorectal deformations.

Notwithstanding the above, there is still a tremendous amount that is still not known about the nature of the mechanisms involved in the etiology of fetal malformations and how to appropriately intervene to reduce or prevent the occurrence of such defects.

### OBJECT OF THE INVENTION

It is therefore an object of the invention to provide a method of treatment of women pre-pregnancy to prevent or reduce the chance of fetal malformations by administering D-chiro-inositol or a phosphate derivative thereof.

It is another object of the invention to provide a method of treatment of women during the first trimester of pregnancy to prevent or reduce the chance of fetal malformations by administering D-chiro-inositol or a phosphate derivative thereof.

It is another object of the invention to provide co-therapy for women pre-pregnancy with both a folate source and D-chiro-inositol or a phosphate derivative thereof.

It is another object of the invention to provide a method of treatment of women during the first trimester of pregnancy to prevent or reduce the chance of fetal malformations by co-administering D-chiro-inositol or a phosphate derivative thereof and a folate source.

It is yet another object of the invention to treat women who are taking birth control pills but who might nonetheless become pregnant by including D-chiro-inositol (or a phosphate thereof) and optionally a folate source into the pills that do not contain an estrogenic substance.

It is yet another object of the invention to treat women who are taking birth control pills but who might nonetheless become pregnant by including D-chiro-inositol (or a phosphate thereof) and optionally a folate source into each of the pills in the birth control pill packet.

It is yet another object of the invention to treat women who are taking birth control pills and who may have estrogen sensitive breast tissue by including D-chiro-inositol (or a phosphate thereof) and optionally a folate source into each of the pills in the birth control pill packet.

It is still another object of the invention to treat women who are on estrogenic hormone therapy and who may have estrogen sensitive breast tissue by administering as co-therapy with said estrogenic hormone therapy D-chiro-inositol (or a phosphate thereof).

It is still another object of the invention to treat women who are on estrogenic hormone therapy and who may have estrogen sensitive breast tissue by administering as a single composition said estrogenic hormone therapy dug and D-chiro-inositol (or a phosphate thereof).

It is still another object of the invention to treat women who are on anti-androgenic hormone therapy and who may have estrogen sensitive breast tissue by administering as co-therapy with said anti-androgenic hormone therapy D-chiro-inositol (or a phosphate thereof).

It is still another object of the invention to treat women who are on anti-androgenic hormone therapy and who may have estrogen sensitive breast tissue by administering as a single composition said anti-androgenic hormone therapy dug and D-chiro-inositol (or a phosphate thereof).

It is still another object of the invention to treat men who are on estrogenic hormone therapy and who may have estrogen sensitive breast tissue by administering as co-therapy with said estrogenic hormone therapy D-chiro-inositol (or a phosphate thereof).

It is still another object of the invention to treat men who are on estrogenic hormone therapy and who may have estrogen sensitive breast tissue by administering as a single composition said estrogenic hormone therapy dug and D-chiro-inositol (or a phosphate thereof).

It is still another object of the invention to treat men who are on anti-androgenic hormone therapy and who may have estrogen sensitive breast tissue by administering as co-therapy with said anti-androgenic hormone therapy D-chiro-inositol (or a phosphate thereof).

It is still another object of the invention to treat men who are on anti-androgenic hormone therapy and who may have estrogen sensitive breast tissue by administering as a single composition said anti-androgenic hormone therapy dug and D-chiro-inositol (or a phosphate thereof).

It is still a further object of the invention to reduce or prevent fetal malformation occurrence where the fetal malformation is a neural tube defect, a cranio-facial defect, an anorectal malformation, caudal regression, etc.

Still further objects of the invention will be apparent to those of ordinary skill.

### SUMMARY OF THE INVENTION

The foregoing fetal malformation prevention objects and others are achieved by treating women of child bearing years with D-chiro-inositol (and/or a phosphate thereof) and optionally a folate source, optimally from pre-conception through at least the first trimester of pregnancy. Inclusion of the D-chiro-inositol along with birth control pills has the added benefit that stores of D-chiro-inositol (and/or phosphates thereof) and folate are high in women taking birth control pills even before they discontinue such treatment or become pregnant notwithstanding being on such therapy. A further benefit of such inclusion is that D-chiro-inositol (or a phosphate thereof) also down-regulates estrogen sensitive receptors in estrogen sensitive breast tissue. The beast cancer avoidance objects of the invention are achieved by administering D-chiro-inositol (with or without folate) to patients who are known to have or are suspect of having breast tissue that is sensitive to estrogenic substance exposure or to anti-androgenic therapy (which may ultimately result in estrogenic excess). The breast cancer avoidance objects of the invention can be achieved in both men and women.

### BRIEF DESCRIPTION OF THE DRAWING

Not Applicable

### DETAILED DESCRIPTION OF THE INVENTION

The present invention is a method of treatment so as to avoid or reduce the incidents of fetal malformations and the avoidance or reduction of activation of breast cancer (or breast cancer precursor condition) in either men or women which men or women are on estrogenic hormonal therapy or anti-androgenic hormonal therapy, which results in an estrogenic/androgenic balance of surplus of estrogenic effects.

D-chiro-inositol is a compound of the structure I

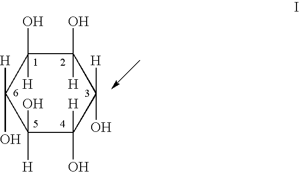

D-chiro inositol is not present in dietary sources and any such compound available to the body must be made by conversion of other sources, either systemically or artificially. The most common source of inositols is myo-inositol, which does occur in dietary sources. Myo-inositol differs from D-chiro-inositol by inversion of the OH and H at the position indicated by the arrow in FIG. 1 above. Methods of making d-chiro-inositol are detailed in a number of patents, among them, U.S. Pat. No. 5,091,596; U.S. Pat. No. 5,406,005; U.S. Pat. No. 5,463,142; U.S. Pat. No. 5,714,643, U.S. Pat. No. 5,932,774; and U.S. Pat. No. 6,660,891, all of which are incorporated herein by reference. Phosphates thereof for purposes of the present invention include those having one or more of the hydroxyl groups in formula I above phosphorylated. These include mono-, di-, tri-, tetra-, penta-, and hexa-phosphates. For convenience, the phosphates of D-chiroinositol will be referred to herein by the term D-chiroIPₓ, where x refers to the number of phosphorylated hydroxyl groups are present. Where there is one or more numbers present as in 1,2-D-chiroIP₂, the designation indicates the position of the phosphate(s) based on the position numbering in FIG. 1 above. A designation such as 1,2-D-IP₃ indicates that positions 1 and 2 are phosphorylated and that another position is phosphorylated, but that it can be at any other position. The absence of any numerical designation before the “IP” indicates that the phosphate groups are not restricted to any particular position(s). The use of the term “IP” without the designation “D-chiro” shall mean that inositol phosphates more generally and include phosphorylated forms of any isomeric form of inositol. Specific mention of particular isomeric forms of inositol, such as myo-, or scyllo-, epi-, etc with the “IPₓ” designation shall refer only to that particular inositol isomer phosphorylated in accordance with the numeric prefix and “x” designation in the foregoing convention. Thus, the present invention relates to compositions and methods of use of D-chiroinositol, its monophosphates (D-chirolP₁), diphosphates (D-chiroIP₂), triphosphates (D-chiroIP₃), tetraphosphates (D-chiroIP₄), pentaphosphates (D-chiroIP₅), and hexaphosphate (D-chiroIP₆). Due to the plane of

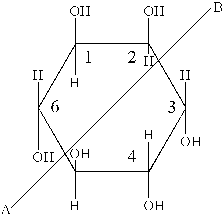

symmetry running along line AB above, D-chiroinositol has 3 distinct monophosphates, 9 distinct diphosphates, 10 distinct triphosphates, 9 distinct tetraphosphates, 3 distinct pentaphosphates, and 1 hexaphosphate, each of which are intended to be included within the scope of the present invention (unless otherwise noted or the context compels otherwise). These are 1-D-chirolP₁, 2-D-chirolP₁, 5-D-chirolP₁, 1,2-D-chiroIP₂, 1,3-D-chiroIP₂, 1,4-D-chiroIP₂, 1,5-D-chiroIP₂, 1,6-D-chiroIP₂, 2,3-D-chiroIP₂, 2,5-D-chiroIP₂, 2,6-D-chiroIP₂, 5,6-D-chiroIP₂, 1,2,3-D-chiroIP₃, 1,2,4-D-chiroIP₃, 1,2,5-D-chiroIP₃, 1,2,6-D-chiroIP₃, 1,3,5-D-chiroIP₃, 1,3,6-D-chiroIP₃, 1,4,5-D-chiroIP₃, 1,5,6-D-chiroIP₃, 2,3,5-D-chiroIP₃, 2,5,6-D-chiroIP₃, 1,2,3,4-D-chiroIP₄, 1,2,3,5-D-chiroIP₄, 1,2,3,6-D-chiroIP₄, 1,2,4,5-D-chiroIP₄, 1,2,4,6-D-chiroIP₄, 1,2,5,6-D-chiroIP₄, 1,3,5,6-D-chiroIP₄, 1,4,5,6,-D-chiroIP₄, 2,3,5,6-D-chiroIP₄, 1,2,3,4,5-D-chiroIP₅, 1,2,3,5,6-D-chiroIP₅, 1,2,4,5,6-D-chiroIP₅, and 1,2,3,4,5,6-D-chiroIP₆. In addition to these phosphates, the invention also includes the corresponding pyrophosphates where at least one of the phosphorylated hydroxyl groups is phosphorylated by a pyrophosphate, such as without limitation compounds such as

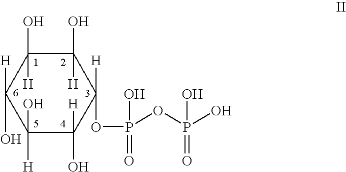

which would be 3-pyrophosphatidyl D-chiroinositol. For simplicity, a pyrophosphatidyl group will be indicated as “PP”, and longer phosphate chains will be designated as “Poly(y)P”, where y indicates the number of phosphate groups in the chain, and y is generally not more 4, but typically 3. Any of the free hydroxyl groups of the D-chiroinositol structure can be phosphorylated with either a single phosphate group, a pyrophosphate group or a longer polyphosphate chain of 3 or more phosphate groups and different hydroxyl groups in the same molecule can be phosphorylated with a variety of these. Thus, for example, without limitation, 1-monophosphatidyl-2-monopyrophosphatadiyl-D-chiroinositol is also within the scope of the invention, as is 1,2-di(monophosphatidyl)-3,4-diPP-5-Poly(3)P-D-chiroinositol. When sterically possible, two hydroxyl groups of the D-chiroinositol structure (within the same molecule can be linked together through a single phosphate group, PP, or Poly(y)P group forming a ring structure or two or more D-chiroinositol molecules can be linked through such phosphate groups as in the non-limiting structure III:

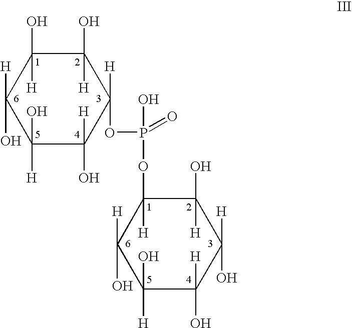

which exemplifies (but does not limit the invention to) a molecule in which two D-chiroinositol molecules are linked through a single phosphate group between position 3 of one D-chiro-inositol and position 1 of the other. The linking phosphate may be a single phosphate, a PP, or Poly(y)P group, and when two or more hydroxyl groups on the same D-chiroinositol structure are phosphorylated, longer chains of alternating D-chiroinositol and a phosphate (single phosphate, PP or Poly(y)P and mixtures thereof) are realized. Further, a phosphate or a pyrophosphate may link two hydroxyl groups as for example, without limitation, in structure IV below:

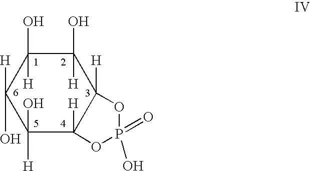

or in a more complex ring structure such as that of formula V below

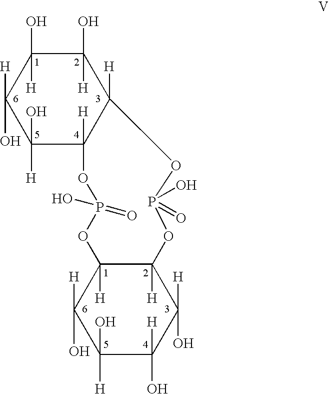

or the two remaining phosphate hydroxyl groups can be dehydrates to form a P—O—P link as well. Each of these more complex D-chiroinositol structures are also within the scope of the present invention. Manufacture of the compounds having PP or Poly(y)P as the phosphorylating group (whether or not linking multiple D-chiroinositol units together) can be prepared in an analogous fashion to the chemical synthesis of the phosphorylates that have only single phosphate groups for any one hydroxyl group by using pyrophosphate of Poly(y)P phosphate chains as the phosphorylation group source.

Formulations in the literature containing chiro-inositol, inositol-phosphates, etc., include, but are not limited to, those disclosed in U.S. Pat. No. 5,124,360; U.S. Pat. No. 5,614,510; U.S. Pat. No. 5,760,222; and U.S. Pat. No. 6,784,209, all of which are incorporated herein by reference in their entirety. Formulations of the D-chiro inositols of the invention and their phosphorylated, pyrophosphated, and polyphosphated derivatives as indicated as being useful in the present invention can be made analogously.

Folic acid and folates are well known in the art as are various formulations thereof. Any of the recognized folates or folic acid is suitable for use in the present invention embodiments that include a folic acid and/or folate component.

Women of child bearing age frequently are avoiding pregnancy by utilizing birth control pills. These are typically estrogenic substances that are administered for a time period and then either stopped for a short time, continued at altered dosage, and/or supplemented or replaced by progestogenic substances so as to induce menses. During the time frame when the estrogenic substance is reduced or stopped, it is possible for a woman to become pregnant. On occasion, it is also possible that the intended “birth control” function of the birth control pills (even when containing a full complement of the estrogenic substance) may not be totally efficacious, such as when other medications or other substances are ingested that interfere with the proper workings of the birth control medication. In such situations a pregnancy may result despite being on such medications. Although there is a general awareness among pregnant women to have proper supplementation with folic acid, many women taking birth control medication do not take adequate supplements of folic acid or many other nutrients that are important to fetal development, simply because they believe that do not need to be concerned with a pregnancy at that time. Others are simply unaware of the need for adequate supplementation, and still others, even though educated about this either neglect to take appropriate supplements or still don't care. Others do not bother because of economic reasons. One aspect of the present invention is to include supplemental D-chiroinositol (and/or one of its phosphorylated derivatives) into birth control pills which may further have folic acid (or other appropriate folate source) incorporated into some or all of the pills in the birth control pill package so as to assure that the woman taken such birth control has adequate stores of D-chiroinositol (and folate, when folate is also incorporated) in the event that she becomes pregnant either while taking birth control pills or during the time period when she initially stops the birth control pill regimen. This is extremely important since both D-chiroinositol and folate are most effective against the various fetal defects that the present invention is directed toward preventing when these substances are administered pre-conception through the first trimester of pregnancy. The D-chiroinositol (and phosphorylated derivatives) and folic acid (and other folates) can be incorporated into just the tablets of the birth control pill package that have either no other active or have progestogenic but not estrogenic substances or have progesterins and low levels of estrogens present, but preferably are incorporated into all of the tablets. This is suitable because generally the higher estrogenic substance tablets will prevent pregnancy and the remaining tablets will begin administering folic acid and D-chiroinositol (or their counterparts) with the first tablet after the estrogenic tablets. However, it is preferable to have the compounds of the invention in all of the tablets in case of a pregnancy that results from birth control tablet failure or due to interference with proper action of the estrogenic substance due to drug interactions or other dietary or environmental impacts that cause birth control failure.

Another aspect of the invention is a combination product having both D-chiroinositol (and/or a phosphorylated (either P, PP, and/or polyP) derivative thereof) and folic acid (and/or other folate source) in a single composition as a nutritional supplement that is especially suited for women of child bearing age who are not yet pregnant (but generally intending to become pregnant), women who are not pregnant and not intentionally trying too become pregnant, but may be, and women who are pregnant. Such fixed combinations may be a standalone product or have other nutritional supplements (or other active agent) incorporated therein. Such additional nutritional supplements include vitamins and minerals as well as herbal products and are well known (both as to substances and their respective dosages) to those of ordinary skill in the nutritional supplement area. Without being held to theory, it is the inventors belief and understanding that co-therapy of folic acid (and/or other folate sources) together with D-chiroinositol (and/or phosphorylated derivatives (P, PP and/or polyP) thereof), whether simultaneously or sequentially, operate in a manner that provides the protective effects against fetal malformations beyond those achievable with either component alone, and further that such results are surprisingly better than those achieved with each alone or that would have been predicted as additive effect. As such, such co-therapy is also within the scope of the present invention, whether such co-therapy is via a fixed combination of folic acid (and/or other folate) and D-chiroinositol (and/or phosphorylated derivates (P, PP and/or polyP) thereof) or via separate administration of these agents generally within 12 hours of each other and generally on a daily basis. Fractional dosing of either or both components taken multiple times a day (i.e., for example ½ daily doses taken twice daily or ⅓ daily dosing taken three times daily) is also within the scope of the present invention. Fractional dosing multiple times a day is particularly suitable when the composition contains only nutritional supplements as active agents and when the patient finds that singe daily doing upsets the stomach or the daily dose is large and not suitable for inclusion into a single unit dosage form.

An additional benefit of administering D-chiroinositol to women on estrogenic medications is the downregulating effect of D-chiroinoisitol of breast tissue sensitivity to estrogenic insult. Thus, incorporation of D-chiroinositol (and/or its phosphorylated (P, PP and/or polyP) derivatives) into fixed combinations with estrogenic medications is a means to increase the safety of the use of estrogenic substances. While D-chiroinositol (and/or its phosphorylated (P, PP and/or polyP) derivatives) can be used as separate medications or nutritional supplements in co-therapy with the estrogenic medication, it is highly preferred to have the D-chiroinositol as a fixed combination with the estrogenic substance as to assure patient compliance. While estrogenic sensitive breast tissue in men is rarer than in women, it does occur and co-therapy in men having estrogenic treatment is also within the scope of the present invention. Furthermore, since estrogenic insult is the result of excess estrogen from endogenous overproduction of estrogen, exogenous administration of estrogen, insufficient androgenic production, or exogenous administration of anti-androgens (androgen ablative therapy), the present invention also includes treating men or women with co-therapy of D-chiroinositol (and/or phosphorylated derivatives (P, PP and/or polyP) thereof) with anti-androgens, which co-therapy can be by separate administration of the compounds of the invention with such anti-androgens or via fixed combinations therewith. The invention still further includes treating patients with conditions that result in excess estrogen (whether because of overproduction of estrogen or insufficient androgen production) with D-chiroinositol (and/or phosphorylated derivatives (P, PP and/or polyP) thereof) as a means of reducing the risk of breast cancer from excess estrogenic insult. Finally, in this group of treatments of the invention, the invention further includes treating patients with a general overproduction of hormonal steroids (even though estrogenic/androgenic balance is maintained). A still further benefit to women who are or become pregnant while receiving the present invention treatment is that of reducing the incidents of gestational diabetes (or if they still do have such, it is a milder case), especially since there has been a connection between gestational diabetes and some fetal malformations. Specific active agents which can be combined for co-therapy with D-chiroinositol (its P, PP, and/or PolyP derivatives) and optionally with addition folic acid (or other folate source) that are within the invention include, without limitation: antiprogestogens, androgens, antiandrogens, estrogens, selective estrogen receptor modulators, aromatase inhibitors, gonadotropins, ovulation stimulators, gonadotropin releasing hormone agonists, gonadotropin releasing hormone antagonists, LHRH agonists, progestins, and anti-progestins, to name a few. Many of these classes are utilized in opposing conditions but the co-therapy with the D-chiroinositol component of the invention and optionally the folate component of the invention is warranted in that in some cases, the effect of the D-chiroinositol component (and optional folate component) is complementary to the other active agent, while in other cases, the D-chiroinositol component (and optional folate component) are protective of one or more of the potential side effects of the other active agent. Specific compounds belonging to these classes of other active agents are exemplified in the following non-exclusive, non-limiting list, each agent of which is prepared in its normal known method and utilized in its known dosage, and include without limitation, abarelix, algestone, amadinone, aminoglutethimide, anagestrone, anastrozole, androisoxazole, androstanolone, androstenediol, 4-androstene-3,16,17-trione, bazedoxifene, benorterone, bicalutamide, bolandiol, bolasterone, bolazine, boldenone, bolenol, bolmantalate, buserelin, calusterone, chlormadinone, chlorotrianisene, chorionic gonadotropin, cioteronel, cingestol, clogestone, clomegestone, clometherone, clomifene, clostebol, conjugated estrogens, cyproterone, danazol, delmadinone, deslorelin, desogestrel, detirelix, dienestrol, diethylstilbestrol, dimethisterone, dihydrogestrone, drospirenone, drostanolone, dydrogesterone, epiestriol, epimestrol, epitiostanol, epristeride, equilin, esterified estrogens, estradiol, estrazinol, estriol, estrofurate, estrone, estropipate, ethinylestradiol, ethisterone, ethylestrenol, ethynerone, ethynodiol, etonogestrel, exemestane, fenestrel, finasteride, fluoxymesterone, flurogestone, flutamide, formebolone, formestane, fosfestrol, fulvestrant, furazabol, ganirelin, gestaclone, gestadienol, gestodene, gestonorone (especially gestonorone caproate), gestrinone, gonadorelin, goserelin, haloprogesterone, histrelin, 4-hydroxy-19-nortestosterone, hydroxyprogesterone, ibutamoren, idoxifene, letrozole, leuprolide, leuprorelin, levonorgestrel, lutrelin, lynestrenol, mebolazine, medrogestone, medroxyprogesterone, megestrol, melengestrel, menotropins (especially humegon, pergonal, repronex), mesabolone, mestranol, mesterolone, metandienone, metenolone, methandriol, methenolone, methestrol, methyltestosterone, methynodiol, metribolone, mibolerone, mifepristone, nafarelin, nafoxidine, nandrolone, nilutamide, nitromifene, norboletone, norbolethone, norclostebol, norelgestromin, norethandrolone, norethindrone, norethisterone, norethynodrel, norgestimate, norgestomet, norgestrel, norgestrienone, nylestriol, oxabolone, oxandrolone, oxendolone, oxogestone, oxymesterone, oxymetholone, polyestradiol (especially polyestradiol phosphate), pralmorelin, prasterone, progesterone, quinbolone, quinestrol, quinestradol, quingestanol (especially quingestanol acetate), quingestrone, raloxifene, rismorelin, somalapor, somatrem, somatropin, somenopor, somidobove, stanozolol, stenbolone, sumorelin, tamoxifen, testosterone, tibolone, tigestrol, tiomesterone, topterone, toremifene, trenbolone, trimegestone, trioxifene, triptorelin, urofollitropin, vorozole, zanoterone, and zeranol, among others, each of which includes the pharmaceutically acceptable salts and esters thereof. These are all known compounds with known uses and are used in the normal course for those known indications. The co-therapy of the present invention adds D-chiroinositol (and/or a P, PP, or PolyP derivative thereof) and optionally folic acid (and/or other folate source) thereto, with the amounts of the D-chiroinositol components and folic acid components being as set forth elsewhere herein. The D-chiroinsoitol and optional folate can be separately administered with these other active agents of combined in fixed combinations therewith as may be convenient.

Turning to the fetal malformations that are the main focus of the present invention, fetal development is a very delicate and sensitive process and there are many points at which something can go wrong, resulting in a congenital defect. Defects may occur because of innate genetic defects, inappropriate nutrition limiting a necessary and sometimes critical nutrient, inability to convert a nutrient into the form needed for proper development, and exposure to toxins, infections, and environmental factors that interfere with the proper functioning of the required developmental machinery or supply of the necessary compounds for that machinery to properly function. As such, no treatment will eliminate all such fetal defects or even all occurrences of any one type of fetal defect. Nonetheless, the administration of D-chiroinositol (and/or phosphorylated derivatives (P, PP and/or polyP) thereof) alone or in combination with folic acid (and/or other folate source) during the first trimester of pregnancy, preferably throughout the first trimester of pregnancy, even more preferably from before conception into the first trimester of pregnancy, and most preferably from before conception through at least the end of the first trimester of pregnancy will significantly reduce the frequency of a wide range of fetal defects, above those reported previously for those patients who have not been treated or those patients who have been treated with either of the D-chiroinositol (and/or its phosphorylated (P, PP and/or polyP) derivatives) or with folic acid (or other folate source) alone (where those treatments have been previously studied. The treatment of the present invention further reduces the frequency of these defects as compared to treatment with other forms of inositol (and/or phosphorylated derivatives thereof) where such treatment has been previously studied.

The defects, the frequency of which the present invention is designed to reduce, include, but are not limited to neural tube defects, craniofacial anomalies, caudal malformations, anorectal malformations, among others. These include, but are not limited to (1) dysraphisms which includes, but is not limited to (a) rachischisis (aka spinal dysraphism) such as spina bifida (including, but not limited to spina bifida aperta (aka spinabifida cystica); spinabifida occulta; and occult spinal disorder, among others) and (b) craniorachischisis (aka cranial dysraphism) such as cranium bifida (aka encephalocele or craniocele) each of spina bifida and cranium bifida being of any of the following types meningocele, myelomeningocele, lipomeningocele, and lipomyelomeningocele among others; (c) anencephaly; and (d) chiari malformation; (2) caudal regression syndrome, caudal dysplasia sequence, congenital sacral agenesis; sacral regression and the like; (3) cranio-facial defects such as, without limitation, facial cleft (aka prosopoanoschisis, including without limitation cleft palate, cleft lip, velopharyngeal malformation (including without limitation bifid uvula), etc.); (4) anorectal malformations including, but not limited to (a) imperforate anus, (b) rectoperineal fistula, (c) recto-bladder neck fistula; (d) persistent urogenital sinus, (e) persistent cloaca, etc.; (5) bucket-handle malformation; among others. The ultimate cause of these conditions is can be genetic or environmental, or both. All of these terms are well known in the art. However, for rapid reference, those unfamiliar with these terms are referred (without limitation to the Merck Manual, Eighteenth Edition 2006 and the PDR Medical Dictionary (2000).

Without being bound to theory, the inventor believes that all of these conditions are related to failure of proper mapping sequences during the critical embryonic first trimester. One embryonic mapping sequence that has been identified is the Sonic hedgehog (Shh) gene and some inositol phosphates (and kinases therefore) have been shown to be important in the proper expression of the Shh gene. It is the present inventor's belief that insufficient D-chiroinositol levels (and/or phosphates (P, PP and/or polyP) thereof) interfere with or prevent the proper expression of the Shh gene and the result thereof is improper signaling of proper mapping of the embryonic tissue. Thus, proper supplementation of D-chiroinositol (and/or phosphorylated derivatives (P, PP and/or polyP) thereof) will restore proper signaling and mapping in the embryo at that critical period (if the embryo is one at risk of such improper signaling and mapping) so as to substantially reduce and/or eliminate the risk of the presentation of the above fetal malformations. Since the risk of some of the above conditions have already been shown to benefit from folate supplementation, co-therapy with both D-chiroinositol (and/or its phosphorylated (P, PP and/or polyP) derivatives) and folic acid (and/or another folate source) is the preferred embodiment of the invention. It is also believed that the D-chiroinositol (or one of its phosphorylated (P, PP and/or polyP) derivatives) is the active agent involved in this mechanism and that either other forms of inositol have a weak (or weaker) effect than the D-chiro version and/or that a significant number of the women having children with these malformations have (a) insufficient inositol intake and therefore cannot convert a sufficient amount to the D-chiro form or (b) simply cannot properly convert other inositol forms to the D-chiro variety. In this subpopulation, supplementation with any of the D-chiroinositol and/or its phosphorylated derivatives will serve equally well. A small subpopulation however may have defects in the various kinases involved and thus, the best supplementation would be with the particular phosphorylate that is after the kinase defect. Since finding the specific defect in a particular kinase may not be easily identified in all cases, a separate embodiment of the present invention is to use a mixture of D-chiroinositol and a number of its phosphorylated (P, PP and/or polyP) derivatives so as to be sure that none of the advantages of the present invention are missed in as many patients as possible. A highly preferred embodiment in this case is to use a mixture of D-chiroinositol and at least one member selected from D-chiroinositol-P₍₁₋₆₎.

Dosages of folic acid can vary from about 100 μg to about 2 mg per day, preferably at least about 200 μg per day, more preferably at least 400 μg per day and should preferably be no more than about 1.6 mg per day, more preferably not more than about 1.2 mg per day. Specific pre-natal dosages of folic acid are well known and any of the literature dosages of this component will be suitable, especially 0.4 mg, 0.6 mg, 0.8 mg, 1.0 mg, 1.2 mg, and 1.4 mg for example. Other folate sources beyond folic acid can be used with or instead of folic acid in amounts that appropriate to result in the same folate delivery as the aforementioned folic acid. Combinations of folic acid and other folate sources are administered in appropriate amounts so that the total is equivalent to a folate dose within the above limitations.

D-Chiroinositol doses (calculated on the basis of unphosphorylated D-chiroinositol) range from about 0.05 mg/day to about 60 grams per day, preferably about 0.05 mg/day to about 30 grams per day, preferably about 0.1 mg to about 25 grams/day, more preferably, about 1 mg to about 20 grams/day, still more preferably about 5 mg to about 10 grams per day, even more preferably about 10 mg to about 5 grams per day, yet more preferably about 25 mg to about 2 grams/day, still even more preferably about 20 mg to about 1.8 grams/day. Highly preferred dosages of D-chiroinositol (and its P, PP, and PolyP derivatives) further include, about 10 mg/kg/day to about 500 mg/kg/day; about 100 mg to about 1 gram/day; about 1.2 gram to about 1.8 gram/day; about 500 mg/day; about 500 to about 700 mg/day; about 25 mg/kg/day to about 100 mg/kg/day. Particular daily doses (based on unphosphorylated D-chiroinositol) include: about 0.1 mg, about 0.2 mg, about 0.5 mg, about 0.8 mg, about 1 mg, about 1.25 mg, about 1.5 mg, about 2 mg, about 2.5 mg, about 3 mg, about 5 mg, about 10 mg, about 12.5 mg, about 15 mg, about 20 mg, about 25 mg, about 40 mg, about 50 mg, about 75 mg, about 100 mg, about 125 mg, about 150 mg, about 200 mg, about 250 mg, about 300 mg, about 350 mg, about 400 mg, about 500 mg, about 750 mg, about 800 mg, about 1 g, about 1.2 g, about 1.4 g, about 1.6 g, about 1.8 g, about 2 g, about 2.4 g, about 2.5 g, about 2.75 g, about 3 g, about 3.5 g, about 4 g, about 5 g, about 6 g, about 8 g, about 10 g, about 12 g, about 15 g, about 18 g, about 20 g, about 22.5 g, about 25 g, about 30 g, about 40 g, about 50 g and about 60 g. These, particularly the larger doses may be administered in fractional doses, all at a single time or spread out over the day as may be convenient.

Compositions of the present invention may be single active agent entities that are merely co-administered as described herein or fixed combinations as indicated above. The other components beyond the folic acid (and/or other folate) and the D-chiroinositol (and/or P, PP, and/or PolyP derivative thereof) can be selected from a wide variety of materials. Additional active agents that may be included in or merely co-administered with the above components include those estrogenic and progestogenic substances used in birth control pills, hormone replacement therapy, androgen ablative therapy, etc (including, but not limited to conjugated estrogens, ethinyl estradiol, levonorgestrel, norgestrel, norgestimate, norethidrone, norethidrone acetate, mestranol, ethynodiol diacetate, norelgestromin, etonogestrel, desogestrel, etc). These hormones are currently marketed under the following (non-limiting) trade names: ALESSE, LEVLEN, LO-OVRAL, TRICYCLEN, ORTHOCEPT, ORTHOEVRA, NUVA RING, OVRAL, TRI-LEVLEN, TRIPHASIL, BREVICON, FEMHRT, LOESTRIN, LoOGESTREL, MICROGESTIN, among others. Where the birth control or hormone replacement therapy dosage form is other than an oral dosage form (such as, for example, a transdermal patch (in the case of currently marketed norelgestromin) or a vaginal ring (in the case of currently marketed etonogestrel estradiol)), the invention objectives are achieved with a co-therapy of a suitable dosage form of the folic acid (and/or other folate source) and D-chiroinositol (and/or P, PP, and/or Poly P derivatives thereof). Andogen ablative therapies for which the instant invention can be used include treatment with for example, without limitation, finasteride as well as other known androgen ablative drugs.

Compositions of the present invention in which the D-chiroinositol component and or the folic acid component are the only active agents can be prepared as in or analogously to those set forth in the patents indicated above as being incorporated herein by reference. For compositions that are disclosed therein that have an inositol component, the D-chiroinositol component (and/or P, PP, and/or PolyP derivative thereof) can be used in direct replacement of the other inositol component indicated therein. The folic acid (and/or other folate source) can be incorporated therein by merely replacing a small portion of filler or merely adding the folic acid (and/or other folate source) thereto. Where the references formulation is a folic acid formulation and the dose selected for the D-chiroinositol (and/or P, PP, and/or PolyP derivative thereof) is sufficiently small, the D-chiro inositol (and/or derivative thereof) can be used in place of a portion or all of the filler used in the referenced formulation, or added to it. If larger amounts are needed, then the filler used in the referenced formulations is replaced with the D-chiroinositol (and/or P, PP, and/or PolyP derivative thereof) component if the resulting tablet size is not of concern. If the size of the dosage form is insufficient to accommodate the full dose of the D-chiroinositol (and/or derivative thereof), then either a separate dosage form is used or multiple dosage forms having a fraction of the daily dose is used and the patient will need to take more than 1 dosage form to achieve the daily dosages set forth.

In preferred dosage forms, the D-chiroinositol (and/or P, PP, and/or PolyP derivatives thereof) is substantially free of the other isomers of inositol. In highly preferred embodiments, the D-chiroinositol (and/or the P, PP, and/or PolyP derivatives thereof) and the dosage forms thereof are completely free of the other isomers of inositol as well as their corresponding phosphorylated derivatives. For purposes of the present invention, “substantially free” means not more than about 5% based on the combined D-chiro forms present, more preferably not more than about 2.5%, still more preferably not more than about 1%, most preferably not more than 0.5%. For purposes of the present invention, “completely free of” or “free of” means below the limit of detection of said non-D-chiro forms respectively in common analytical techniques used in common pharmaceutical quality control of bulk materials as of the date of the invention herein.

### EXAMPLES

The following non-limiting Examples are presented only to exemplify various embodiments of the invention and do not limit it in any fashion.

### Example 1

Females having been determined to be at risk of fetal malformations and who are seeking a further pregnancy are split into no treatment, folate treatment, D-chiroinositol treatment, and Folate+D-chiroinositol treatment arms. The respective regimens are administered once daily from before conception through the end of the first trimester. Relative to the untreated controls, the frequency of fetal malformations is reduced in each of the non-control arms. However, the reduction in frequency of fetal malformations in the co-therapy of the present invention is significantly better than in either of the other treatment arms.

### Example 2

Females beginning birth control medication are assigned to similar treatment and control groups as in Example 1. Treatment is begun at the time of initiation of birth control medication, and continued until after a pregnancy occurs and for the following first trimester of pregnancy. Similar reductions as reported in Example 1 are seen. In addition, follow up of these females shows a lower level of breast cancer development than expected.

### Example 3

Men preparing to initiate androgen ablative therapy are initiated on a course of D-chiroinositol prior to and throughout the treatment with the androgen ablative therapeutic. The frequency of male breast cancer found in these patients is substantially reduced as compared to controls not receiving the D-chiroinositol therapy.

1. A method for the prevention of or reducing the risk of birth defects comprising administering to a female of child bearing years a co-therapy comprising a first therapy of a component (a) which is a D-chiroinositol component selected from the group consisting of (i) D-chiroinositol, (ii) at least one phosphate of D-chiroinositol having (iia) from 1 to 6 monophosphate groups per molecule, (iib) 1-6 pyrophosphate groups per molecule, (iic) 1-6 polyphosphate groups per molecule, (iid) cyclic derivatives of the forgoing wherein one or more phosphate groups together with the D-chiroinositol ring to which they are attached form at least one phospho containing ring, (iii) mixtures thereof, (iv) pharmaceutically acceptable salts thereof, and (v) mixtures thereof; and a second therapy of a component (b) comprising folic acid, one or more non-folic acid folate sources, pharmaceutically acceptable salts thereof and mixtures thereof.

2. The method of claim 1 wherein said component b is selected from the group consisting of folic acid or a pharmaceutically acceptable salt thereof, and mixtures thereof.

3. The method of claim 1 wherein said component b is administered in an amount equivalent to about 200 μg to about 1.6 mg of folic acid per day.

4. The method of claim 3 wherein said component b is administered in an amount equivalent to an amount of folic acid selected from about 200 μg, about 250 μg, about 300 μg, about 350 μg, about 400 μg, about 450 μg, about 500 μg, about 600 μg, about 650 μg, about 700 μg, about 750 μg, about 800 μg, about 850 μg, about 900 μg, about 950 μg, about 1 mg, about 1.05 mg, about 1.1 mg, about 1.15 mg, about 1.2 mg, about 1.25 mg, about 1.3 mg, about 1.35 mg, about 1.4 mg, about 1.45 mg, about 1.5 mg, about 1.55 mg, and about 1.6 mg per day.

5. The method of claim 1 wherein said component a is selected from 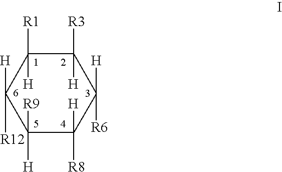
where each or R1, R3, R6, R8, R9, and R10 is independently OH or —O{P(═O) (OH)O}ₙP(OH)₂(═O) in which n is 1-3, a poly D-chiroinositolphosphorylate of the following structure: 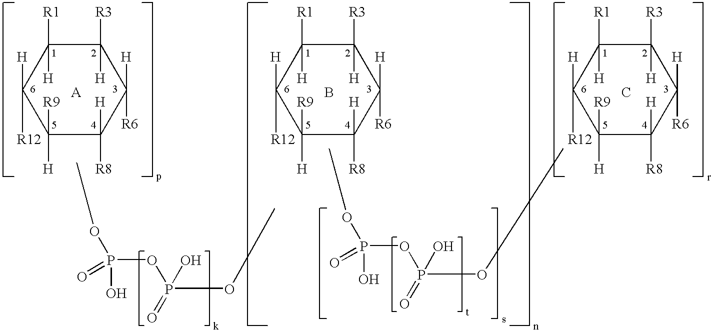
where each of the groups R1, R3, R6, R8, R9, and R12, in each unit are independently as set forth above except that one of such R groups in each of the A and C structures is a direct bond to the indicated oxygen instead of the foregoing, and one of such R groups in each B structure is a direct bond to one of the two indicated oxygens instead of the above and a second of the R groups in each B structure is a direct bond to the other indicated oxygen, p, r, and s are each 1, t and k are each independently an integer of from 0 to 2, and n is a an integer of from 0 to 8; pharmaceutically acceptable salts thereof, and mixtures thereof.

6. The method of claim 1 wherein said component (a) is administered in an amount which is the same molar amount as of from about 0.1 mg/day to about 60 g per day of D-chiroinositol.

7. The method of claim 6 wherein said component a is administered in an amount that is equivalent to an amount of D-chiroinositol selected from the group consisting of about 0.1 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 50 mg, about 75 mg, about 100 mg, about 125 mg, about 150 mg, about 200 mg, about 250 mg, about 300 mg, about 350 mg, about 400 mg, about 500 mg, about 600 mg, about 700 mg, about 750 mg, about 800 mg, about 900 mg, about 950 mg, about 1 g per day, about 1.2 g per day, about 1.8 g per day, about 2 g per day, about 2.5 g per day, about 3 g per day, about 5 g per day, about 10 g per day, about 12 g per day, about 18 g per day, about 24 g per day, about 30 g per day, about 45 g per day, and about 60 g per day.

8. The method of claim 1 wherein said composition further comprises a member selected from the group consisting of an estrogenic substance, a progestogenic substance, and combination thereof.

9. The method of claim 1 wherein said combination is a distinct formulation, free from hormonally active agents packaged together with at least one separate second formulation, said second formulation comprising at least one member selected from (a) at least one estrogenically active agent, (b) at least one progestogenically active agent, and (c) combinations thereof.

10. The method of claim 9 wherein said package is dispensed for an indication of birth control.

11. The method of claim 1 wherein said birth defect is selected from the group consisting of at least one of (a) neural tube defects, (b) craniofacial anomalies, (c) anorectal malformation, (d) caudal malformation, and (e) combinations thereof.

12. A method of reducing or preventing breast tissue sensitivity to estrogenic insult (a) from dietary or environmental or medicinal sources in a patient in need thereof and/or (b) a patient of greater than average risk of such sensitivity comprising administering to said patient an estrogenic sensitivity reducing amount of a member selected from the group consisting of D-chiroinositol, a D-chiroinositol phosphate, and mixtures thereof.

13. The method of claim 12 wherein said insult is from medicinal sources.

14. The method of claim 13 wherein said patient is a female receiving at least one treatment selected from birth control, hormonal replacement therapy, or antiandrogenic therapy; or said patient is a male receiving at least one treatment selected from estrogenic treatment and hormonal ablative therapy; or said patient is a male to female trans-sexual receiving at least one therapy selected from estrogenic treatment and antiandrogenic treatment.

15. A composition comprising (a) a first component selected from folic acid, a non-folic acid folate source, pharmaceutically acceptable salts thereof, and mixtures thereof; and (b) a second component selected from the group consisting of (1) D-chiroinositol, (2) one or more phosphorylated derivatives of D-chiroinositol, (3) pharmaceutically acceptable salts thereof, and (4) mixtures thereof.

16. The composition of claim 15 further comprising at least one component selected from the group of consisting of (a) at least one estrogenic material, (b) at least one progestogenic material, (c) at least on antiandrogenic material, and (d) combinations thereof.

17. The composition of claim 15 being free of any estrogenic active agent, progestogenic active agent, and antiandrogenic active agent, and being packaged together with at least one second formulation, said second formulation comprising at least one active agent selected from the group consisting of (a) at least one estrogenic material, (b) at least one progestogenic material, (c) at least one antiandrogenic material, and (d) combinations thereof.

18. A composition comprising a component (i) selected from the group consisting of (a) D-chiroinositol, and (b) at least one phosphate of D-chiroinositol having from (b1) 1 to 6 monophosphate groups per molecule, (b2) 1-6 pyrophosphate groups per molecule, (b3) 1-6 polyphosphate groups per molecule, (b4) cyclic derivatives of the forgoing wherein one or more phosphate groups together with the D-chiroinositol ring to which they are attached form at least one phospho containing ring, and (c) mixtures thereof and a component (ii) comprising at least one component selected from the group of consisting of (a) at least one estrogenic material, (b) at least one progestognic material, (c) at least one antiandrogenic material, and (d) combinations thereof.

19. The method of claim 1 wherein said co-therapy is administered via a fixed combination of at least one component a compound and at least one component b compound.

20. A method of reducing or preventing breast tissue sensitivity to estrogenic insult from estrogenic, progestogenic, or antiandrogenic therapy in patient receiving such therapy comprising administering to said patient co-therapy therewith using a an estrogenic sensitivity reducing amount of at least one compound selected from the group consisting of D-chiroinositol, a D-chiroinositol phosphorylated derivative, pharmaceutically acceptable salts thereof and mixtures thereof.

21. A co-therapy method comprising administering
(a) D-chiroinositol or a P, PP, or Poly P derivative thereof,
(b) optionally folic acid or another folate source, and
(c) one or more agents selected from the groups of classes of active agents consisting of antiprogestogens, androgens, antiandrogens, estrogens, selective estrogen receptor modulators, aromatase inhibitors, gonadotropins, ovulation stimulators, gonadotropin releasing hormone agonists, gonadotropin releasing hormone antagonists, LHRH agonists, progestins, and anti-progestins.

22. The method of claim 21 wherein at least one compound with claim 21 (a) and at least one agent within claim 21 (c) are in fixed combination.

23. A fixed combination for use in the method of claim 21 wherein at least one compound with claim 21 (a) and at least one agent within claim 21 (c) are in fixed combination.

24. The fixed combination of claim 23 wherein the agent of claim 22 (c) is selected from the group consisting of abarelix, algestone, amadinone, aminoglutethimide, anagestrone, anastrozole, androisoxazole, androstanolone, androstenediol, 4-androstene-3,16,17-trione, bazedoxifene, benorterone, bicalutamide, bolandiol, bolasterone, bolazine, boldenone, bolenol, bolmantalate, buserelin, calusterone, chlormadinone, chlorotrianisene, chorionic gonadotropin, cioteronel, cingestol, clogestone, clomegestone, clometherone, clomifene, clostebol, conjugated estrogens, cyproterone, danazol, delmadinone, deslorelin, desogestrel, detirelix, dienestrol, diethylstilbestrol, dimethisterone, dihydrogestrone, drospirenone, drostanolone, dydrogesterone, epiestriol, epimestrol, epitiostanol, epristeride, equilin, esterified estrogens, estradiol, estrazinol, estriol, estrofurate, estrone, estropipate, ethinylestradiol, ethisterone, ethylestrenol, ethynerone, ethynodiol, etonogestrel, exemestane, fenestrel, finasteride, fluoxymesterone, flurogestone, flutamide, formebolone, formestane, fosfestrol, fulvestrant, furazabol, ganirelin, gestaclone, gestadienol, gestodene, gestonorone (especially gestonorone caproate), gestrinone, gonadorelin, goserelin, haloprogesterone, histrelin, 4-hydroxy-19-nortestosterone, hydroxyprogesterone, ibutamoren, idoxifene, letrozole, leuprolide, leuprorelin, levonorgestrel, lutrelin, lynestrenol, mebolazine, medrogestone, medroxyprogesterone, megestrol, melengestrel, menotropins, mesabolone, mestranol, mesterolone, metandienone, metenolone, methandriol, methenolone, methestrol, methyltestosterone, methynodiol, metribolone, mibolerone, mifepristone, nafarelin, nafoxidine, nandrolone, nilutamide, nitromifene, norboletone, norbolethone, norclostebol, norelgestromin, norethandrolone, norethindrone, norethisterone, norethynodrel, norgestimate, norgestomet, norgestrel, norgestrienone, nylestriol, oxabolone, oxandrolone, oxendolone, oxogestone, oxymesterone, oxymetholone, polyestradiol, pralmorelin, prasterone, progesterone, quinbolone, quinestrol, quinestradol, quingestanol, quingestrone, raloxifene, rismorelin, somalapor, somatrem, somatropin, somenopor, somidobove, stanozolol, stenbolone, sumorelin, tamoxifen, testosterone, tibolone, tigestrol, tiomesterone, topterone, toremifene, trenbolone, trimegestone, trioxifene, triptorelin, urofollitropin, vorozole, zanoterone, zeranol, and mixtures thereof.
